(19) United States
(12) Patent Application Publication
(10) Pub. No.: US 2022/0267249 A1
(43) Pub. Date: Aug. 25, 2022

(54) Method for Preparation of Decolorized Acetoacetylated Ethylene Glycol

(71) Applicant: Arxada AG, Visp (CH)

(72) Inventors: Christophe Girard, St-Leonard (CH); Maximilian Kuehnle, Rheinfelden (CH)

(21) Appl. No.: 17/626,837

(22) PCT Filed: Jul. 17, 2020
(86) PCT No.: PCT/EP2020/070335

§ 371 Date: Jan. 13, 2022

(30) Foreign Application Priority Data
Jul. 17, 2019 (EP) 19186766.2

(51) Int. Cl. C07C 67/54

(52) U.S. Cl. CPC C07C 67/54

(57) ABSTRACT

The invention discloses the use of a short path evaporator for decolorization of colorized acetoacetylated ethylene glycol.

The invention discloses the use of a short path evaporator for decolorization of colorized acetoacetylated ethylene glycol.

### BACKGROUND OF THE INVENTION

U.S. Pat. No. 5,459,178 A discloses the use of acetoacetylated ethylene glycol (AAEG) as a component of a foundry binder which is used for the preparation of foundry shapes. The foundry binder comprises an acetoacetate ester such as AAEG, an alpha, beta ethylenically unsaturated monomer; and a liquid tertiary amine catalyst.

AAEG may be prepared by diketeneization of ethylene glycol; the product of the diketeneization is a colorized AAEG. The color of the colorized AAEG remains in the foundry binder and in the foundry shapes made from the foundry binder.

A. F. Joslyn et al. Dimeric 1,4-dihydropyridines as calcium channel antagonists. J Med Chem. 1988; 31(8):1489-1492 discloses the synthesis of 1,n-alkanediylbis(1,4-dihydropyridines) (n=2, 4, 6, 8, 10, 12) bridged at C₃ of 2,6-dimethyl-3-carboxy-5-carbethoxy-4-(3-nitrophenyl)-1,4-dihydropyridine.

There was a need for an AAEG with less color than provided by the diketeneization in order to be able to produce less colorized foundry shapes.

Commonly used distillation apparatuses such a wiped thin film evaporators are not capable of decolorizing colorized AAEG.

Surprisingly a short path evaporator is capable of decolorizing colorized AAEG, decolorized AAEG can thereby be obtained.

Abbreviations and Definitions

AAEG acetoacetylated ethylene glycol

Ex example

wt % weight %, percent by weight

### SUMMARY OF THE INVENTION

Subject of the invention is a method for preparation of decolorized acetoacetylated ethylene glycol by a distillation of colorized acetoacetylated ethylene glycol with a short path evaporator.

### DETAILED DESCRIPTION OF THE INVENTION

The short path evaporator is working continuously. The colorized AAEG is fed continuously into the short path evaporator.
The short path evaporator has a jacket, a wiped film evaporator and an internal condenser.
The distillation may be done at a temperature of from 100 to 250° C., preferably of from 110 to 230° C., more preferably of from 120 to 210° C., even more preferably 130 to 190° C., especially from 140 to 170° C.; preferably, the temperature is the temperature of the jacket of the short path evaporator.
The distillation may be done at a vacuum of from 0.01 to 5 mbar, preferably of from 0.05 to 2.5 mbar, more preferably of from 0.1 to 1 mbar.
Any value of a vacuum is herein meant to be an absolute value, if not stated explicitly otherwise.
The short path evaporator splits the feed into a distillate, which is the product, the decolorized AAEG, and into a residue, which contains the color.
The split ratio is defined in percent as [(weight of distillate)/(weight of residue+weight of distillate)].
The split ratio may be from 20 to 99.9%, preferably from 30 to 99.8%, more preferably from 40 to 99.7%, even more preferably from 50 to 99.6%, especially from 55 to 99.5%, more especially from 55 to 99%.
The split ratio is set by the chosen feed flowrate in combination with the specific geometry of the used short path evaporator and the chosen temperature and vacuum.
The colorized AAEG may be degassed before it is fed into the short path evaporator.
Any degassing may be done at a temperature of from 100 to 200° C., preferably of from 110 to 190° C., more preferably of from 120 to 180° C., even more preferably 130 to 170° C., especially from 140 to 160° C.
Any degassing may be done at a vacuum of from 3 to 100 mbar, preferably of from 3 to 50 mbar, more preferably of from 3 to 25 mbar, even more preferably from 3 to 10 mbar.
Any degassing may be done for 1 sec to 2 h.

### EXAMPLES

Materials
short path evaporator VKL 38-1 of VTA Verfahrenstechnische Anlagen GmbH & Co. KG, 94559 Niederwinkling, Germany, area 0.01 m², inner diameter 38 mm

Methods

The CIELAB and Hazen values were determined according to ISO 11664 (CIELAB) and ISO 6271 (APHA/Hazen) with a Thermo Scientific Evolution 220 of Fisher Scientific GmbH, 58239 Schwerte, Germany, using a 10 mm rectangular cuvette. The method limits for the Hazen value were 50 to 200.

### Examples 1 to 3

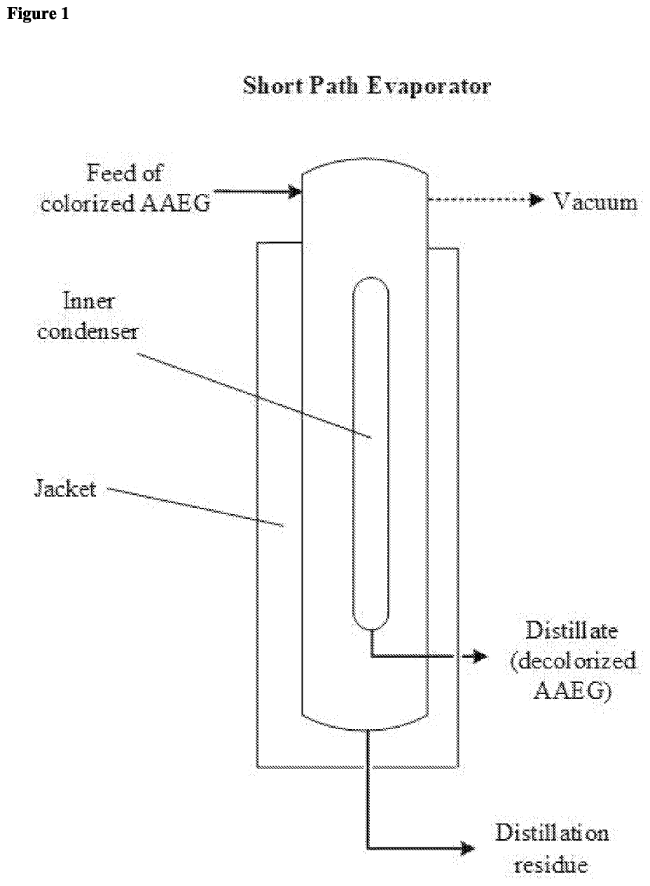

A short path evaporator was used as shown in FIG. 1 to decolorize colorized AAEG, prepared according to example 4. The short path evaporator had a wiped film evaporator with an internal condenser. The jacket temperature was set to 150° C. and the vacuum was set to 0.5 mbar.

Colorized AAEG, prepared according to example 4, was degassed at 150° C. and 5 mbar for 1 h, and was then continuously fed into the short path evaporator. The distillate contained the decolorized AAEG, while a colored residue was discarded. Details are given in Table 1. The split ratio was set as given in Table 1 by choosing a respective feed flowrate.

TABLE 1

| | Split | | Jacket | Color | Color | Color | Color |
|---|---|---|---|---|---|---|---|
| | ratio | Pressure | T | CIELAB | CIELAB | CIELAB | APHA |
| Ex | [%] | [mbar] | [° C.] | L* | a* | b* | Hazen |
| AAEG(*) | | | | 82.9 | 29.3 | 118 | above |
| | | | | | | | method |
| | | | | | | | limits |
| 1 | 67 | 0.5 | 150 | 99.9 | 0 | 1.9 | 67 |
| 2 | 98 | 0.5 | 150 | 99.6 | −0.2 | 4.4 | 157 |
| 3 | 88 | 0.5 | 150 | 99.6 | 0.1 | 1.6 | 55 |

(*)Colorized AAEG before distillation, prepared according to example 4

### Example 4

### Preparation of Colorized AAEG

700 g Ethylene glycol and 2.3 g triethylamine were heated to 60° C. under stirring. 1850 g Diketene were dosed within 6 h under stirring providing a reaction mixture in form of a solution. Then the reaction mixture was stirred for 1 h at 60° C., then the reaction mixture was cooled to 20° C. Colorized AAEG with a content according to NMR of 92.6 wt %, based on the weight of the sample, was obtained.

### Comparative Example 1

A lab glass wiped thin film evaporator was tested to distill and decolorize colorized AAEG prepared according to example 4. The jacket temperature was set to 180° C. and the vacuum was set to 10 mbar. The feeding rate to the lab glass wiped thin film evaporator was 200 g/hr. Under these conditions the sample did not boil and therefore no separation or decolorization could be achieved.

1. A method for preparation of decolorized acetoacetylated ethylene glycol comprising distillation of colorized acetoacetylated ethylene glycol (AAEG) with a short path evaporator.

2. The method according to claim 1, wherein the short path evaporator is working continuously.

3. The method according to claim 1, wherein the colorized AAEG is fed continuously into the short path evaporator.

4. The method according to claim 1, wherein the distillation is done at a temperature of from 100 to 250° C.

5. The method according to claim 1, wherein the distillation is done at a vacuum of from 0.01 to 5 mbar.

6. The method according to claim 1, wherein
the split ratio is from 20 to 99.9%;
with the split ratio being defined in percent as [(weight of distillate)/(weight of residue+weight of distillate)].

7. The method according to claim 1, wherein the colorized acetoacetylated ethylene glycol degassed before it is fed into the short path evaporator.

8. The method according to claim 7, wherein any degassing is done at a temperature of from 100 to 200° C.

9. The method according to claim 7, wherein any degassing is done at a vacuum of from 3 to 100 mbar.
